(19) United States
(12) Patent Application Publication
(10) Pub. No.: US 2018/0228388 A1
(43) Pub. Date: Aug. 16, 2018

(54) BREATHING FUNCTION EXAMINING INSTRUMENT

(71) Applicant: DENSO CORPORATION, Kariya-city, Aichi-pref. (JP)

(72) Inventors: Shinya KUROSAWA, Kariya-city (JP); Rie OSAKI, Kariya-city (JP); Kazuo TOKUSHIMA, Kariya-city (JP)

(21) Appl. No.: 15/749,603

(22) PCT Filed: Jul. 12, 2016
(86) PCT No.: PCT/JP2016/070550

§ 371 Date: Feb. 1, 2018

(30) Foreign Application Priority Data
Aug. 6, 2015 (JP) 2015-155904

(51) Int. Cl. A61B 5/03; A61B 5/087

(52) U.S. Cl. CPC A61B 5/038; A61B 5/087

(57) ABSTRACT

A main body portion is shaped in a cylinder and formed with an intake port which is a hole through which air from an outside flows into the cylinder. The main body portion has one end, which is provided with one-way valve to impede inflow of air from the outside, and the other end, which is not provided with the one-way valve and through which the subject breathes. The resistance setting unit variably sets a magnitude of an inflow resistance to air flowing into the main body portion. The pressure measurement unit measures a pressure of air moving in the cylinder of the main body portion as the intraoral pressure.

### CROSS REFERENCE TO RELATED APPLICATION

This application is based on Japanese Patent Application No. 2015-155904 filed on Aug. 6, 2015, the disclosure of which is incorporated herein by reference.

### TECHNICAL FIELD

The present disclosure relates to a breathing function examining instrument to measure an intraoral pressure.

### BACKGROUND ART

Conventionally, an intrathoracic pressure estimation device provided with a pulse wave acquisition unit that acquires a pulse wave signal representing a pulse wave of a subject and an estimation unit that estimates an intrathoracic pressure of the subject based on the pulse wave signal acquired with the pulse wave acquisition unit has been known (refer to Patent Literature 1).

The estimation unit of the intrathoracic pressure estimation device disclosed in Patent Literature 1 creates a first envelope, which connects peaks of the pulse wave of one beat represented by the pulse wave signal, and creates a second envelope that connects peaks of the first envelope. The estimation unit estimates a difference between the first envelope and the second envelope as an intrathoracic pressure signal representing an intrathoracic pressure of the subject.

### PRIOR TECHNICAL LITERATURE

### Patent Literature

PATENT LITERATURE 1: JP-A-2002-355227

Incidentally, the intrathoracic pressure signal estimated with the intrathoracic pressure calculation device disclosed in Patent Literature 1 represents a relative transition of a pressure, and indicates a relative value of the intrathoracic pressure. In order to convert a relative value of the intrathoracic pressure to an absolute value, calibration is required.

The calibration is implemented by multiplying the intrathoracic pressure signal by a calibration coefficient. The calibration coefficient is a coefficient based on a correspondence relationship between an intraoral pressure of a subject and an intrathoracic pressure signal. The calibration coefficient is obtained in advance based on a correspondence relationship between pulse wave signals of the subject measured at the time of breathing and the intraoral pressure of the subject, by causing the subject to perform breathing with different depths.

As a method of measuring the intraoral pressure of the subject in breathing with different depths in advance, it is conceivable that the subject performs breathing at each prescribed ventilation amount and the intraoral pressure is measured with a sensing mechanism provided in a breathing function examining instrument.

However, when the subject performs breathing at each predetermined ventilation amount, it is difficult to adjust the breathing of the subject so as to produce the different ventilation amount. As a result, in the conventional technique, there is a fear that an issue would arises that the subject cannot perform the breathing with different depths, and consequently making it difficult to obtain the calibration coefficient.

In other words, it is conceivable that a technique that enables the subject to perform the breathing with different depths in a simpler manner is effective.

### SUMMARY OF INVENTION

### Technical Issue

It is an object of the present disclosure to produce a technique that enables a subject to perform breathing with different depths in a simpler manner.

According to one aspect of the present disclosure, a breathing function examining instrument to measure an intraoral pressure of a subject which changes with breathing comprises a main body portion. The main body portion is formed in a cylinder shape, has an intake port as a hole through which air from an outside is to flow into the cylinder, has one end provided with a one-way valve, which is to impede inflow of air from the outside, and has an other end, which is not provided with the one-way valve and through which the subject is to breathe. The breathing function examining instrument further comprises a resistance setting unit to variably set a magnitude of an inflow resistance to air, which is to flow into the main body portion. The breathing function examining instrument further comprises a pressure measurement unit to measure, as the intraoral pressure, a pressure of air moving in the cylinder of the main body portion.

### BRIEF DESCRIPTION OF THE DRAWINGS

The above and other objects, features and advantages of the present disclosure will become more apparent from the following detailed description made with reference to the accompanying drawings. In the drawings:

FIG. 1 is a perspective view illustrating a breathing function examining instrument according to a first embodiment;

FIG. 2A is a top view of a breathing function examining instrument according to the first embodiment, and FIG. 2B is a cross-sectional view taken along a line IIB-IIB of FIG. 2A;

FIG. 3 is a graph showing a range of resting breathing;

FIG. 4A is a top view of a breathing function examining instrument according to one modification of the first embodiment, and FIG. 4B is a cross-sectional view taken along a line IVB-IVB of FIG. 4A;

FIG. 5A is a top view of a breathing function examining instrument according to another modification of the first embodiment, and FIG. 5B is a cross-sectional view taken along a line VB-VB of FIG. 5A;

FIG. 6A is a top view of a breathing function examining instrument according to a second embodiment, and FIG. 6B is a cross-sectional view taken along a line VIB-VIB of FIG. 6A;

FIG. 7A is a perspective view showing one of fitting portions, and FIG. 7B is a perspective view showing a fitting portion different from that in FIG. 7A;

FIG. 8A is a top view of a breathing function examining instrument according to a modification of the second embodiment, and FIG. 8B is a cross-sectional view taken along a line VIIIB-VIIIB of FIG. 8A; and

FIG. 9A is a top view of a breathing function examining instrument according to a third embodiment, and FIG. 9B is a cross-sectional view taken along a line IXB-IXB of FIG. 9A.

### DESCRIPTION OF EMBODIMENTS

Hereinafter, the embodiments of the present disclosure will be described with reference to the drawings.

### First Embodiment

<Breathing Function Examining Instrument>

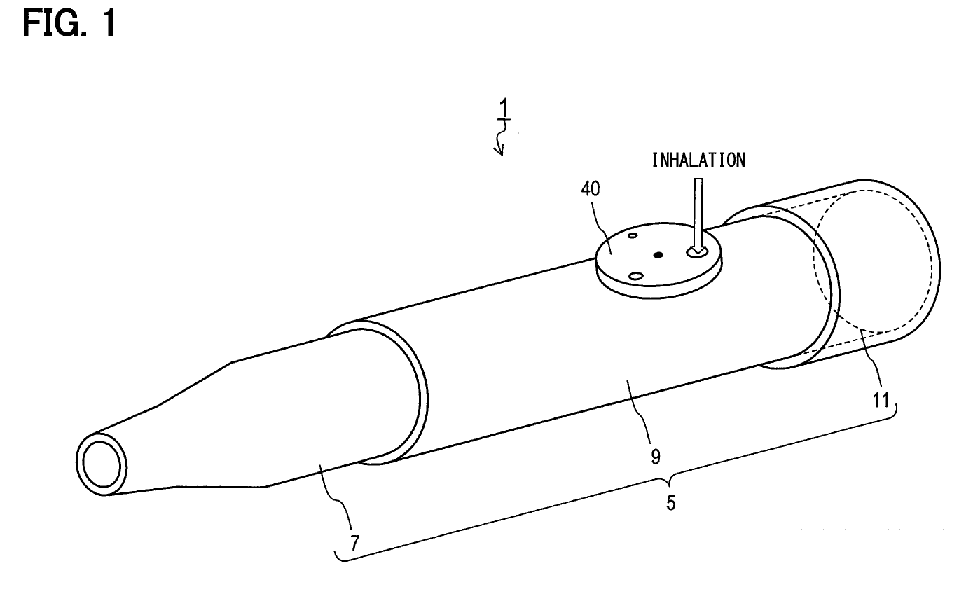

A breathing function examining instrument 1 shown in FIG. 1 is a device for measuring an intraoral pressure of a subject. The intraoral pressure measured with the breathing function examining instrument 1 is used for calculating a calibration coefficient for calculating an absolute value of an intrathoracic pressure of the subject based on a pulse wave signal representing a pulse wave of the subject.

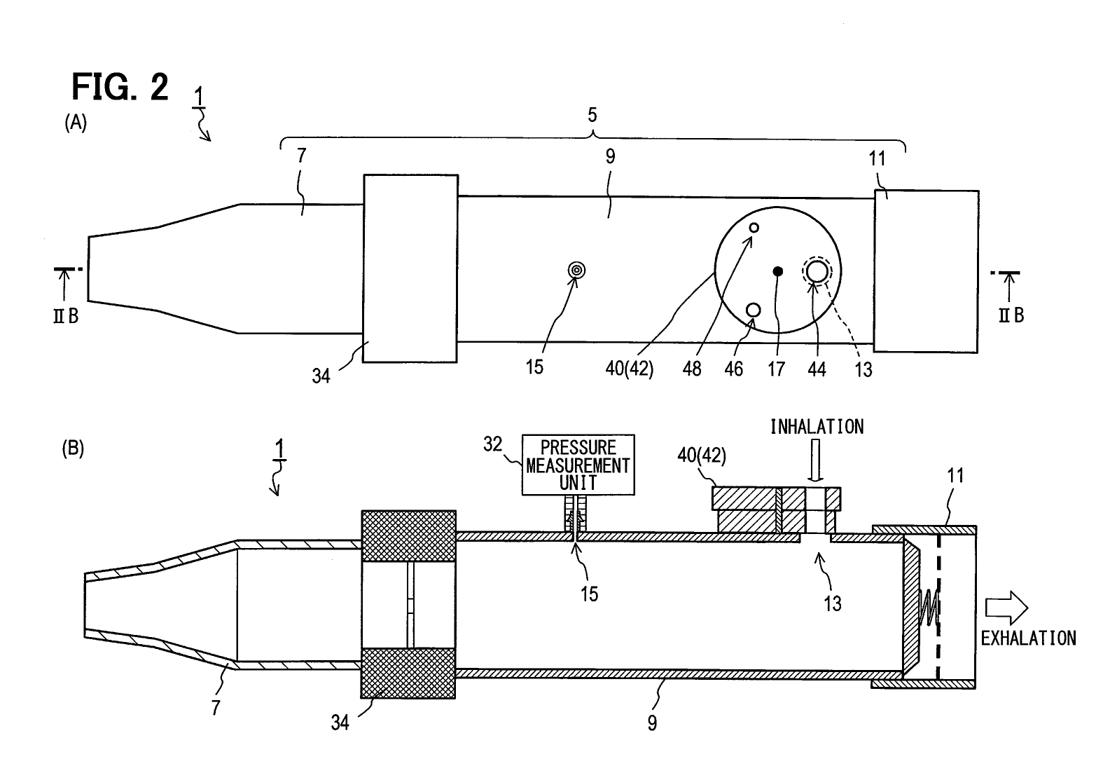

The breathing function examining instrument 1 includes a main body portion 5, a pressure measurement unit 32 (refer to FIG. 2B), a flow rate measurement unit 34 (refer to FIG. 2B), and a resistance setting unit 40. As shown in FIGS. 2A and 2B, the main body portion 5 is a cylindrical member and includes a mouthpiece 7, a main body cylindrical portion 9, and a one-way valve 11.

The main body cylindrical portion 9 is a cylindrical member. An intake port 13 and a pressure measurement hole 15 are provided in the main body cylindrical portion 9. The intake port 13 is a hole through which an air flows into the main body cylindrical portion 9 from an outside. The pressure measurement hole 15 is a hole for measuring a pressure of an air flowing in the main body cylindrical portion 9. A pressure measurement unit 32 is connected to the pressure measurement hole 15

In other words, in the main body cylindrical portion 9, an inspiration which is an air sucked by the subject and an expiration which is an air discharged by the subject flow in the cylinder. The one-way valve 11 is a valve device that is fixed to one end of the main body cylindrical portion 9. The one-way valve 11 restricts an air from the outside from flowing into the main body cylindrical portion 9 and allows the expiration flowing through the main body cylindrical portion 9 to flow out to the outside.

The mouthpiece 7 is a cylindrical member. The mouthpiece 7 according to the present embodiment is connected to an end of the main body cylindrical portion 9 where the one-way valve 11 is not provided. The inspiration which is an air sucked by the subject and the expiration which is an air discharged by the subject flow in the mouthpiece 7.

The pressure measurement unit 32 measures a pressure of the air moving in the cylinder of the main body portion 5 by one breathing of the subject as an intraoral pressure. Measurement of the intraoral pressure according to the present embodiment is carried out continuously along a time axis. It is conceivable to use a well-known pressure sensor as the pressure measurement unit 32 in the embodiment.

Further, the flow rate measurement unit 34 measures a flow rate of the air moving in the cylinder of the main body portion 5 by one breathing of the subject as the ventilation amount. Measurement of the ventilation amount according to the present embodiment is carried out continuously along a time axis. It is to be noted that a ventilation amount referred to in the present specification is the amount of air flowing by one breathing, that is, the respiration amount. In the embodiment, it is conceivable to use a well-known flow rate sensor as the flow rate measurement unit 34.

<Resistance Setting Unit>

The resistance setting unit 40 is configured to be capable of changing a magnitude of a resistance to the air (that is, expiration) flowing into the main body cylindrical portion 9. The resistance setting unit 40 according to the present embodiment covers at least a part of the intake port 13 provided in the main body cylindrical portion 9 to change the magnitude of an inflow resistance. The inflow resistance referred to in the present specification is a resistance that restricts the flow of air from the outside into the main body cylindrical portion 9.

The resistance setting unit 40 according to the present embodiment includes an adjustment plate 42 that is formed in a circular plate-shape. One or more vent holes 44, 46, and 48 are pierced in the adjustment plate 42. The vent holes 44, 46, and 48 penetrate through the adjustment plate 42. The vent holes 44, 46, and 48 are smaller in diameter than the intake port 13 provided in the main body cylindrical portion 9, and are different in diameter from each other.

The diameter of the vent hole 48 having the smallest diameter among the vent holes 44, 46, and 48, that is, an upper limit value of the inflow resistance is determined as the upper limit value at which the subject can breathe at the resting breathing at a prescribed ventilation amount. The resting breathing referred to in this example is breathing performed by only contraction and relaxation of a breathing muscle, not so-called effort breathing.

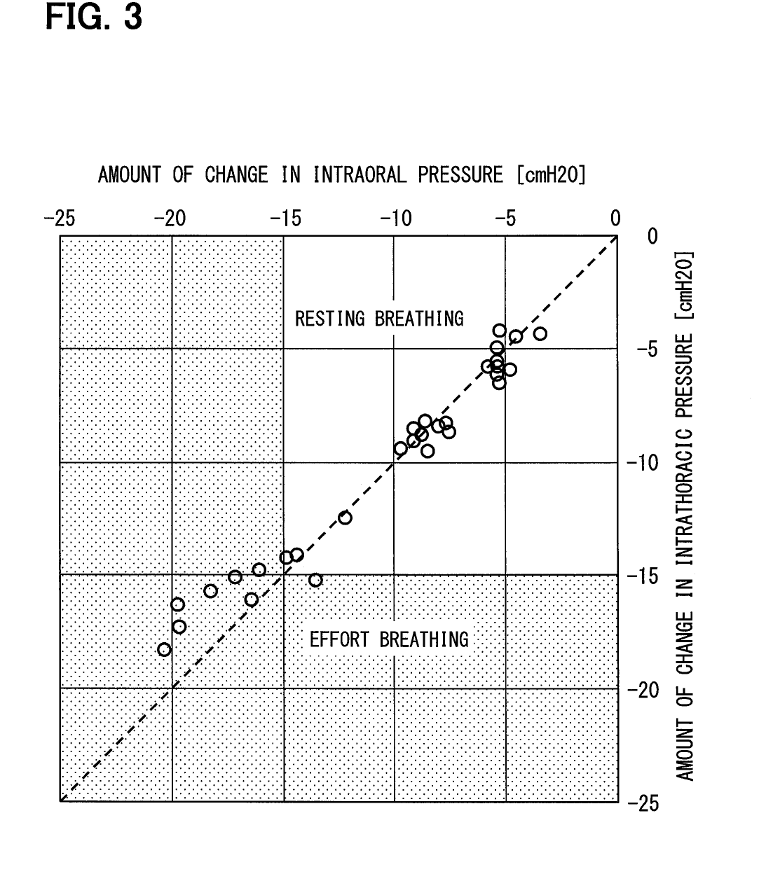

As an example of a resting breathing range, as shown in FIG. 3, the amount of change in the intraoral pressure ranges from 0 cm H2O to −15 cm H2O. As an example of the upper limit value at which breathing can be performed at resting breathing, −15 cm H2O is conceivable. The reason why the above range and the upper limit value are appropriate is as follows. As a result of intensive research by the present inventors, the present inventors have obtained a knowledge that the amount of change in the intraoral pressure of the subject from a first reference value is equal to the amount of change in the intrathoracic pressure from a second reference value, regardless of the magnitude of a resistance between an oral cavity and a thoracic cavity.

The first reference value referred to in the present specification is a preset value of the intraoral pressure. As an example of the first reference value, intraoral pressure at an end of expiration is considered. The second reference value referred to in the present specification is a preset value of the intrathoracic pressure. As an example of the second reference value, the intrathoracic pressure at an end of inspiration is considered.

In other words, based on the above knowledge, a correspondence relationship between a variation of the amount of change in the intraoral pressure from the first reference value and a variation of the amount of change in an estimated intrathoracic pressure from the second reference value is derived as a calibration coefficient. When an absolute value of the intrathoracic pressure of the subject is calculated by multiplying the estimated intrathoracic pressure by the derived calibration coefficient, a calculation accuracy of the absolute value of the intrathoracic pressure can be improved.

In addition, the estimated intrathoracic pressure referred to in the present specification is an estimation value of the intrathoracic pressure which is estimated based on a pulse wave signal representing a pulse wave of the subject. A method of calculating the estimated intrathoracic pressure is well known as disclosed, for example, in JP-A-2002-355227, and therefore a detailed description of the calculation method will be omitted.

By the way, the adjustment plate 42 is rotatably supported by a support protrusion 17 that protrudes from the main body cylindrical portion 9 toward the outside of the main body cylindrical portion 9. The “rotatably” referred to in the present specification includes a configuration in which the adjustment plate 42 is rotatable with respect to the main body portion 5 in such a manner that an area covering the intake port 13 can be changed.

In the other words, in the resistance setting unit 40, the adjustment plate 42 is rotated so that peripheral edges of the vent holes 44, 46, and 48 provided in the adjustment plate 42 cover a part of the intake port 13 of the main body cylindrical portion 9. With a change in the area covering the intake port 13 when the adjustment plate 42 is rotated, the magnitude of the resistance to the air (in other words, inspiration) flowing into the main body cylindrical portion 9 is changed.

<Operation of Breathing Function Examining Instrument>

The subject performs a prescribed number of breathing in order to measure the intraoral pressure with a resistance different in magnitude with constant ventilation amount.

When the subject performs single breathing with the use of the breathing function examining instrument 1, the subject first sucks an air through the mouthpiece 7 of the breathing function examining instrument 1. Then, an external air flows into the main body cylindrical portion 9 through the resistance setting unit 40 and the intake port 13 of the main body cylindrical portion 9. Then, the air (intake air) flowing into the main body cylindrical portion 9 passes through the mouthpiece 7, passes through the oral cavity of the subject, and moves into the thoracic cavity of the subject.

When the subject expires the air, the air from the subject's thoracic cavity passes through the oral cavity, passes through the mouthpiece 7, and flows into the main body cylindrical portion 9. Further, the air (intake air) flowing into the main body cylindrical portion 9 opens the one-way valve 11 and flows out to the outside.

The pressure measurement unit 32 of the breathing function examining instrument 1 measures a pressure of the air moving in the cylinder of the main body cylindrical portion 9 by one breathing of the subject as an intraoral pressure.

Subsequently, the subject rotates the adjustment plate 42 so as to change the area of the main body cylindrical portion 9 covering the intake port 13. Then, the subject breathes with the use of the breathing function examining instrument 1 while keeping the above breathing and the ventilation amount at the same level. The subject repeats the procedure described above.

The measurement of the intraoral pressure according to the present embodiment is carried out continuously along a time axis during the implementation of the prescribed number of breathing. Further, the flow rate measurement unit 34 of the breathing function examining instrument 1 measures a flow rate of the air moving in the cylinder of the main body cylindrical portion 9 by one breathing of the subject as the ventilation amount. The measurement of the ventilation amount according to the present embodiment may be carried out continuously along the time axis during the implementation of the prescribed number of breathing.

As described above, according to the breathing function examining instrument 1, the adjustment plate 42 is rotated so that the area covering the intake port 13 of the main body cylindrical portion 9 is changed, thereby being capable of changing the magnitude of the inflow resistance which impedes the flow of air into the main body cylindrical portion 9 from the outside. In other words, according to the breathing function examining instrument 1, the magnitude of the inflow resistance can be easily changed.

The inflow resistance of different magnitudes is set as described above and the subject performs the breathing with the constant ventilation amount by a prescribed number of times prescribed in advance, thereby enabling to perform the prescribed number of breathing with different depths. In other words, according to the breathing function examining instrument 1, the subject can perform the breathing having different depths in a simpler manner.

As a result, according to the breathing function examining instrument 1, the intraoral pressure of the subject in breathing having different depths can be easily measured. When an absolute value of the intrathoracic pressure of the subject is calculated by multiplying the estimated intrathoracic pressure by the calibration coefficient derived based on the correspondence relationship between the intraoral pressure measured as described above and the pulse wave signal along the time axis, a calculation accuracy of the absolute value of the intrathoracic pressure can be improved.

By the way, according to the breathing function examining instrument 1, with only the rotation of the adjustment plate 42, the magnitude of the inflow resistance can be changed, and a change in the magnitude of the inflow resistance by the resistance setting unit 40 can be more easily produced.

More particularly, in the breathing function examining instrument 1, when the position of the adjustment plate 42 is set such that the vent holes 44, 46, 48 and the intake port 13 are concentric with each other, the air is taken into a respiratory apparatus of the subject when the subject breathes through the breathing function examining instrument 1.

In the breathing function examining instrument 1 according to the above embodiment, sensing is performed with the pressure measurement unit 32 and the flow rate measurement unit 34 continuously along the time axis during a period of performing the prescribed number of breathing. Therefore, with notification of the result of the sensing, the subject can recognize whether the prescribed number of breathing is appropriate breathing, or not. Then, if the prescribed number of breathing is not appropriate breathing, a breathing mode can be corrected so as to approach the appropriate breathing.

### Modification of First Embodiment

The embodiments of this disclosure have been described above. However, the present disclosure is not limited to the embodiments described above, and various modifications can be implemented without departing from the spirit of the present disclosure.

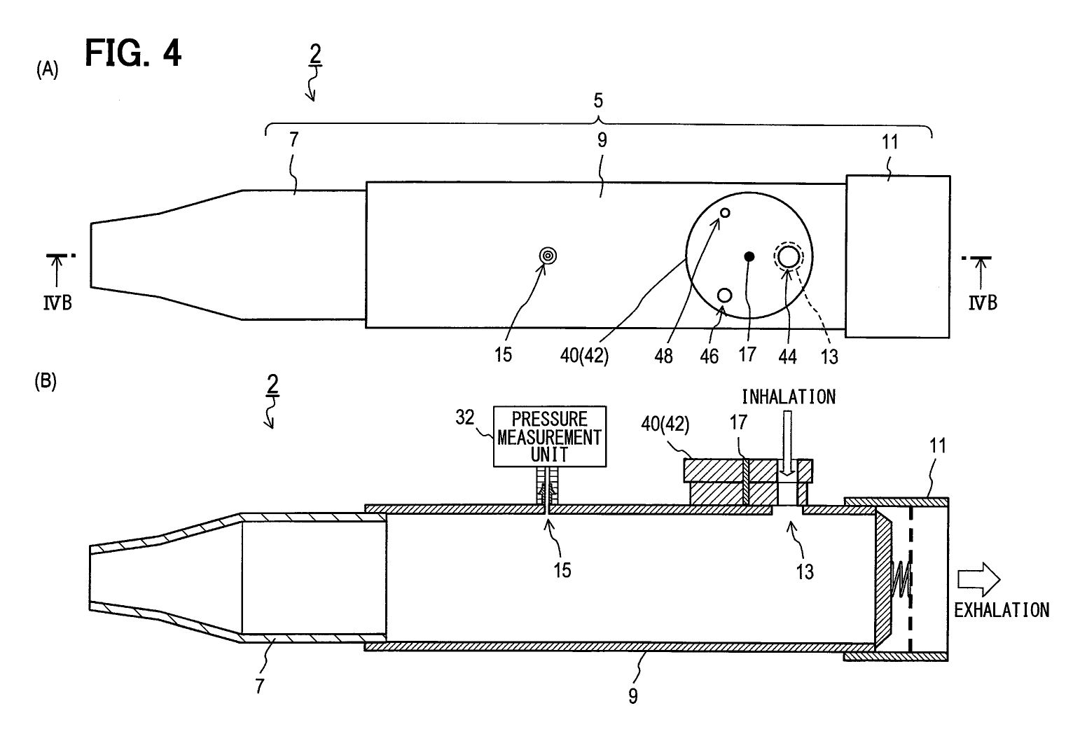

For example, as shown in FIGS. 4A and 4B, the breathing function examining instrument 2 may include a main body portion 5, a pressure measurement unit 32, and a resistance setting unit 40. In other words, in the breathing function examining instrument 2, the flow rate measurement unit 34 may be omitted.

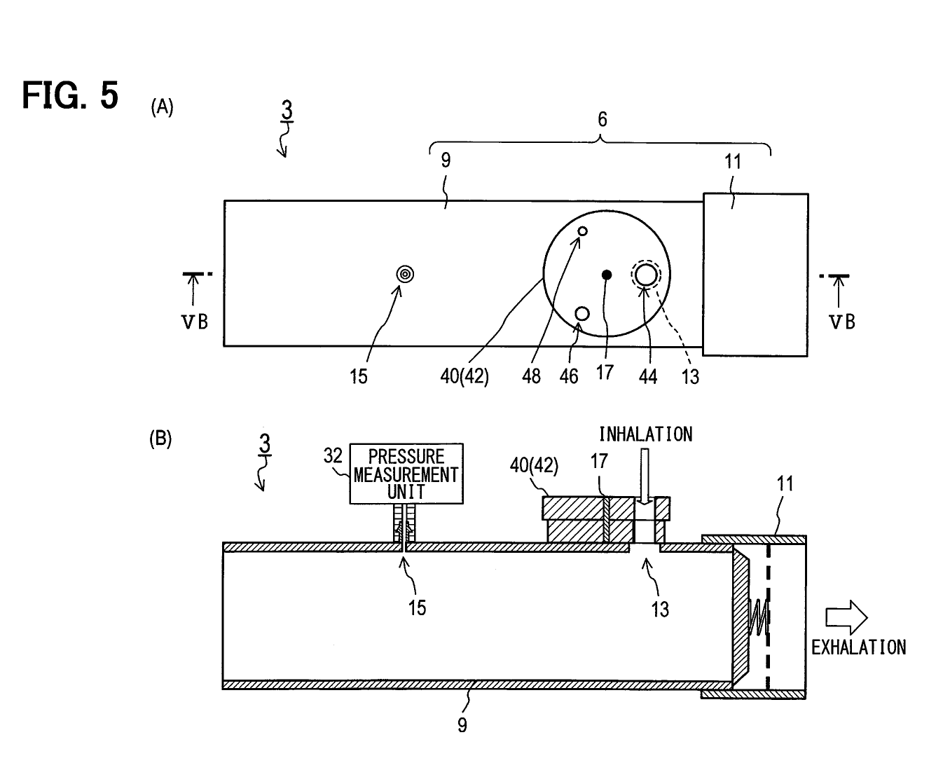

Further, as shown in FIGS. 5A and 5B, a main body portion 6 of the breathing function examining instrument 3 may include a main body cylindrical portion 9 and a one-way valve 11. In other words, in the main body portion 6 of the breathing function examining instrument 3, the mouthpiece 7 may be omitted.

Further, although the adjustment plate 42 in the resistance setting unit 40 of the above embodiment is supposed to be rotated by a person's hand, the adjustment plate 42 may be rotated by a driving force generated by a motor. In this case, when a prescribed number of breathing is implemented in order to measure the intraoral pressure with a resistance of a different magnitude with a constant ventilation amount, the adjustment plate 42 may be rotated so as to produce a resistance of the magnitude prescribed along the time axis.

Furthermore, the adjustment plate 42 according to the above embodiment is pierced with the multiple vent holes, but one vent hole may be pierced in the adjustment plate 42. In the above embodiment, the adjustment plate 42 is configured so that peripheral edges of the vent holes pierced in the adjustment plate 42 cover a part of the intake port 13 of the main body cylindrical portion 9.

This is an example of variably setting the air inflow resistance, and the configuration of the adjustment plate 42 can be selected from various modes.

### Second Embodiment

The breathing function examining instrument according to the second embodiment is different in the configuration of the resistance setting unit from the breathing function examining instrument in the first embodiment. For that reason, the same reference numerals are assigned to the common configurations, a description of the common configurations will be omitted, and the resistance setting unit having a different configuration will be mainly described.

<Breathing Function Examining Instrument>

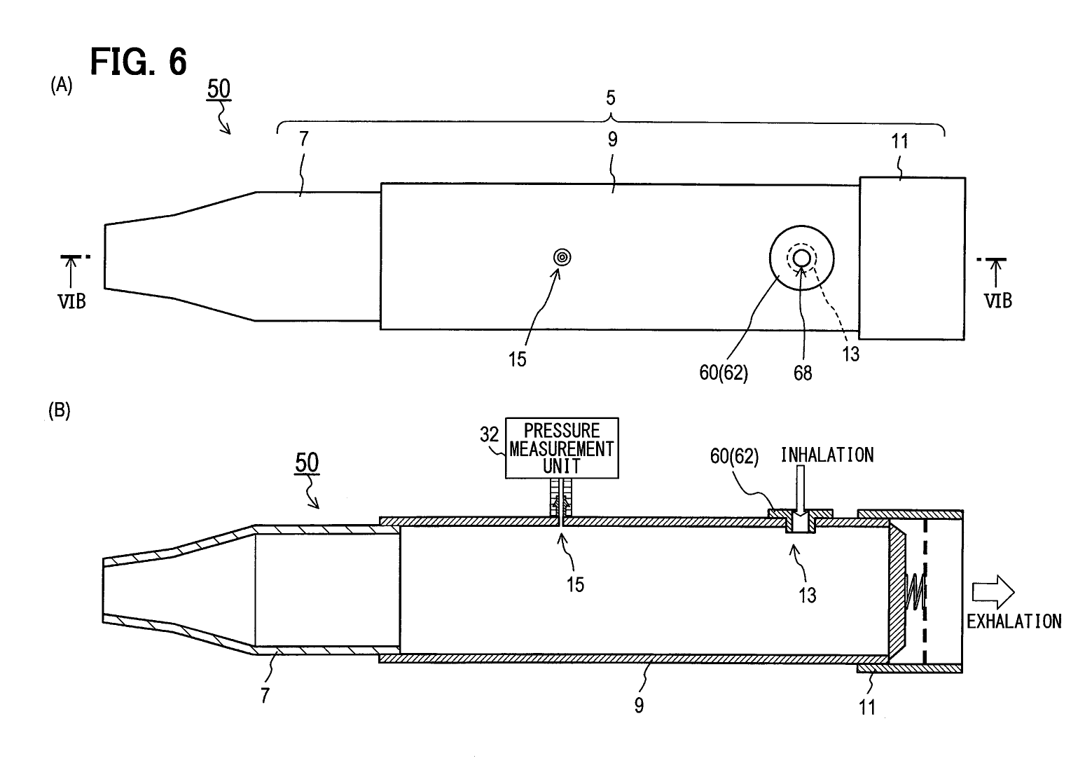

A breathing function examining instrument 50 shown in FIGS. 6A and 6B is a device for measuring an intraoral pressure of a subject. The intraoral pressure measured with the breathing function examining instrument 50 is used for calculating a calibration coefficient for calculating an absolute value of an intrathoracic pressure of the subject based on a pulse wave signal representing a pulse wave of the subject.

The breathing function examining instrument 50 includes a main body portion 5, a pressure measurement unit 32, and a resistance setting unit 60.

<Resistance Setting Unit>

The resistance setting unit 60 is configured to be capable of changing a magnitude of a resistance to the air (that is, expiration) flowing into the main body cylindrical portion 9. The resistance setting unit 60 according to the present embodiment covers at least a part of the intake port 13 provided in the main body cylindrical portion 9 to change the magnitude of an inflow resistance.

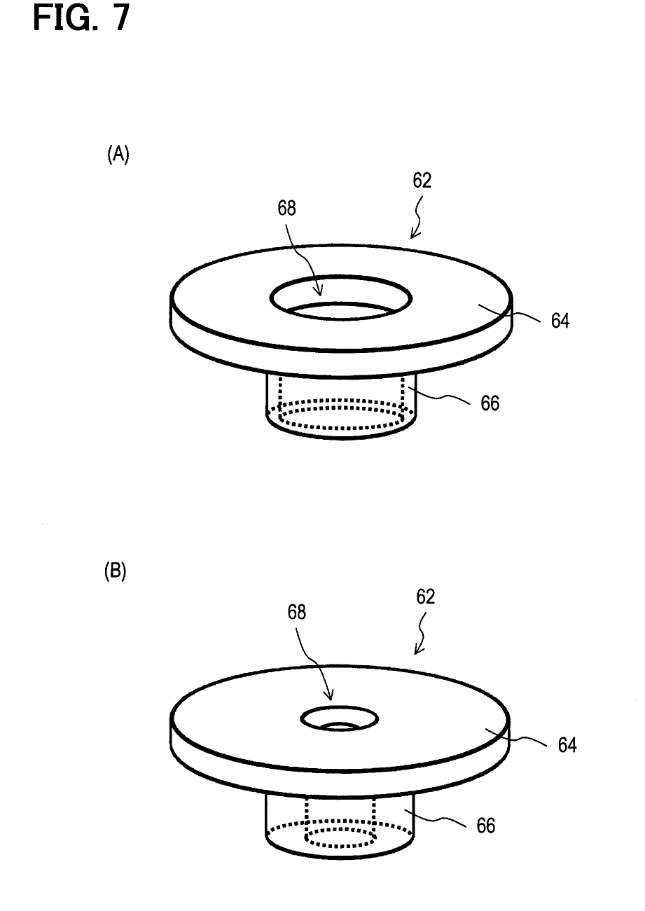

The resistance setting unit 60 according to the present embodiment includes multiple fit portions 62. As shown in FIG. 7A, each of the fitting portions 62 includes a locking portion 64 and an insertion portion 66, and is replaceably attached to the intake port 13.

The locking portion 64 is a disk-shaped portion having a larger diameter than a diameter of the intake port 13 of the main body cylindrical portion 9. The insertion portion 66 is a columnar portion protruding from one surface of the locking portion 64, and is provided concentrically with the locking portion 64. An outer diameter of the insertion portion 66 has a size to be fitted into the intake port 13 of the main body cylindrical portion 9.

Further, in each of the fitting portions 62, a vent hole 68 penetrating through the locking portion 64 and the insertion portion 66 is pierced in the center of the locking portion 64 and the insertion portion 66. The vent hole 68 is smaller in diameter than the intake port 13, and as shown in FIG. 7B, each of the fitting portions 62 has a different diameter. The smallest diameter of the vent holes 68, in other words, an upper limit value of the inflow resistance is determined as an upper limit value at which the subject breathes at the resting breathing with the prescribed ventilation amount.

In the resistance setting unit 60, the fitting portions 62 attached to the intake port 13 of the main body cylindrical portion 9 are replaced with the fitting portions 62 pierced with the vent holes 68 of different diameters, to thereby change the magnitude of the inflow resistance.

<Operation of Breathing Function Examining Instrument>

The subject performs a prescribed number of breathing in order to measure the intraoral pressure with a resistance different in magnitude with constant ventilation amount.

When the subject performs single breathing with the use of the breathing function examining instrument 50, the subject first sucks an air through the mouthpiece 7 of the breathing function examining instrument 50. Then, an air from the outside passes through the vent hole 68 of the resistance setting unit 60, passes through the main body cylindrical portion 9 and the mouthpiece 7, passes through the oral cavity of the subject, and moves into the thoracic cavity of the subject.

When the subject expires the air, the air from the subject's thoracic cavity passes through the oral cavity, passes through the mouthpiece 7, and flows into the main body cylindrical portion 9. Further, the air (intake air) flowing into the main body cylindrical portion 9 opens the one-way valve 11 and flows out to the outside.

The pressure measurement unit 32 of the breathing function examining instrument 50 measures a pressure of the air moving in the cylinder of the main body cylindrical portion 9 by one breathing of the subject as an intraoral pressure.

Subsequently, the subject replaces the fitting portions 62 with another to be attached to the intake port 13 of the main body cylindrical portion 9 so that the diameter of the intake port 13 of the main body cylindrical portion 9, eventually the diameter of the vent hole 68 of the resistance setting unit 60 is changed. Then, the subject breathes with the use of the breathing function examining instrument 50 while keeping the above breathing and the ventilation amount at the same level. The subject repeats the procedure described above.

### Advantages of Second Embodiment

As described above, in the breathing function examining instrument 50, the fitting portion 62 can be replaced with another. When the fitting portion 62 is replaced with another, the magnitude of the inflow resistance can be changed by changing the existing vent holes 68 to the fitting portions 62 that are pierced with the vent holes 68 having different diameters.

As a result, according to the breathing function examining instrument 50, the resistance setting unit 60 capable of changing the magnitude of the inflow resistance can be produced with a simple configuration.

### Modification of second Embodiment

The embodiments of this disclosure have been described above. However, the present disclosure is not limited to the embodiments described above, and various modifications can be implemented without departing from the spirit of the present disclosure.

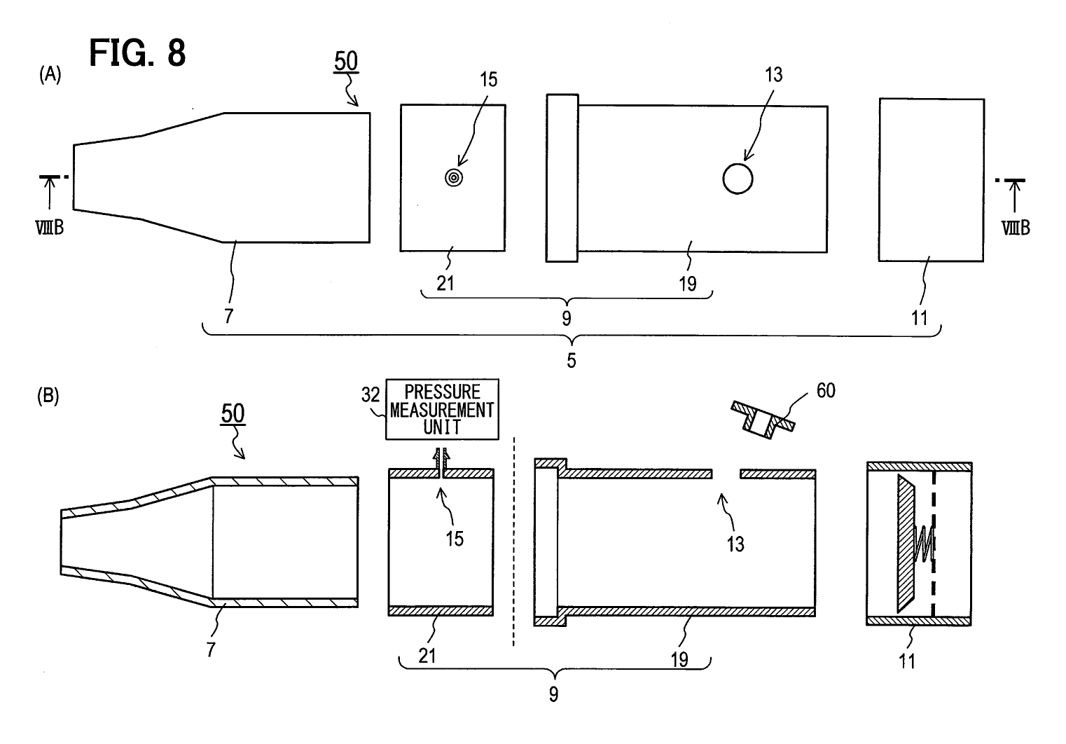

For example, as shown in FIGS. 8A and 8B, the main body cylindrical portion 9 of the breathing function examining instrument 50 may include two members of a first cylinder portion 19 and a second cylinder portion 21. The first cylinder portion 19 referred to in the present specification is a member formed in a cylindrical shape and is a member pierced with the intake port 13. Further, the second cylinder portion 21 is a cylindrical member connected to one end of the first cylinder portion 19, and is a member provided with a pressure measurement hole 15.

### Third Embodiment

A breathing function examining instrument according to a third embodiment is different in the configuration of a resistance setting unit from the breathing function examining instrument 1 in the first embodiment and the breathing function examining instrument 50 in the second embodiment. For that reason, the same reference numerals are assigned to the common configurations, a description of the common configurations will be omitted, and the resistance setting unit having a different configuration will be mainly described.

<Breathing Function Examining Instrument>

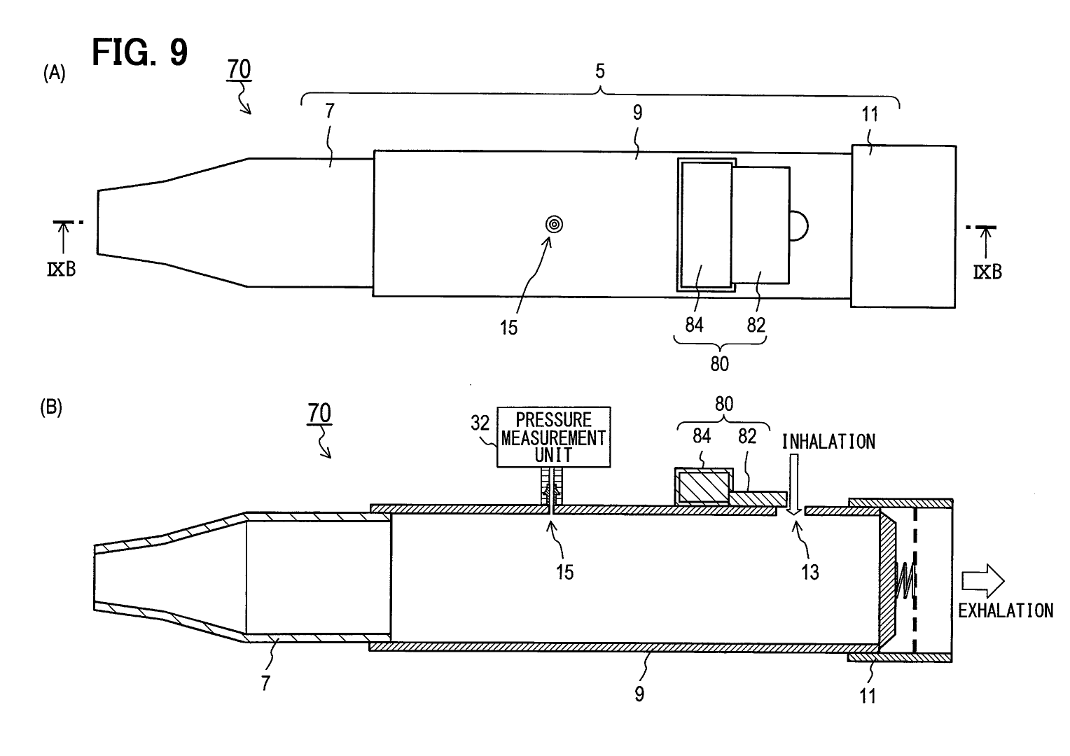

A breathing function examining instrument 70 shown in FIGS. 9A and 9B is a device for measuring an intraoral pressure of a subject. The intraoral pressure measured with the breathing function examining instrument 70 is used for calculating a calibration coefficient for calculating an absolute value of an intrathoracic pressure of the subject based on a pulse wave signal representing a pulse wave of the subject.

The breathing function examining instrument 70 includes a main body portion 5, a pressure measurement unit 32, and a resistance setting unit 80.

<Resistance Setting Unit>

The resistance setting unit 80 is configured to be capable of changing a magnitude of a resistance to the air (that is, expiration) flowing into the main body cylindrical portion 9. The resistance setting unit 80 according to the present embodiment covers at least a part of the intake port 13 provided in the main body cylindrical portion 9 to change the magnitude of an inflow resistance.

The resistance setting unit 80 according to the present embodiment includes an adjustment plate 82 and a housing unit 84. The adjustment plate 82 is a plate-shape member having a larger area than that of the intake port 13 of the main body cylindrical portion 9. The housing portion 84 accommodates the adjustment plate 82.

The resistance setting unit 80 has a structure in which the adjustment plate 82 slides relative to the main body portion 5 so as to close a part of the intake port 13 of the main body cylindrical portion 9. In other words, the adjustment plate 82 is attached to an outer surface of the main body portion 5 so as to move between a covering position for covering at least a part of the intake port 13 of the main body cylindrical portion 9 and an opening position for opening the intake port 13 of the main body cylindrical portion 9.

It is to be noted that the open position referred to in the present specification is a position where the entire adjustment plate 82 is accommodated in the housing portion 84. In the resistance setting unit 80, the adjustment plate 82 is slid to change its area covering the intake port 13 of the main body cylindrical portion 9, thereby changing the magnitude of the inflow resistance.

<Operation of Breathing Function Examining Instrument>

The subject performs a prescribed number of breathing in order to measure the intraoral pressure with a resistance different in magnitude with constant ventilation amount.

When the subject performs single breathing with the use of the breathing function examining instrument 70, the subject first sucks an air through the mouthpiece 7 of the breathing function examining instrument 70. Then, an external air flows into the main body cylindrical portion 9 through the intake port 13 of the main body cylindrical portion 9. Then, the air (intake air) flowing into the main body cylindrical portion 9 passes through the mouthpiece 7, passes through the oral cavity of the subject, and moves into the thoracic cavity of the subject.

When the subject expires the air, the air from the subject's thoracic cavity passes through the oral cavity, passes through the mouthpiece 7, and flows into the main body cylindrical portion 9. Further, the air (intake air) flowing into the main body cylindrical portion 9 opens the one-way valve 11 and flows out to the outside.

The pressure measurement unit 32 of the breathing function examining instrument 70 measures a pressure of the air moving in the cylinder of the main body cylindrical portion 9 by one breathing of the subject as an intraoral pressure.

Subsequently, the subject slides the adjustment plate 82 so as to change the area of the main body cylindrical portion 9 covering the intake port 13. Then, the subject breathes with the use of the breathing function examining instrument 70 while keeping the above breathing and the ventilation amount at the same level. The subject repeats the procedure described above.

### Advantages of Third Embodiment

As described above, according to the breathing function examining instrument 70, the magnitude of the inflow resistance can be changed by only sliding the adjustment plate 82. In other words, the resistance setting unit 80 that changes the magnitude of the inflow resistance can be produced with a simple configuration.

### Other Embodiments

The embodiments of this disclosure have been described above. However, the present disclosure is not limited to the embodiments described above, and various modifications can be implemented without departing from the spirit of the present disclosure.

For example, the breathing function examining instrument 50 according to the second embodiment and the breathing function examining instrument 70 according to the third embodiment do not include the flow rate measurement unit 34. However, the breathing function examining instrument 50 according to the second embodiment and the breathing function examining instrument 70 according to the third embodiment may have the flow rate measurement unit 34.

Further, the main body cylindrical portion 9 may be configured by combining three or more parts together. The main body portion 5 may be configured by one component. In other words, the main body portion 5 of the breathing function examining instrument 50 may be configured by one part, or may be configured by combining at least two or more parts together.

Modes in which a part of the configurations of the above embodiments may be omitted are also encompassed by the embodiments of the present disclosure. Modes configured by appropriate combinations of the above embodiments with the modification are also encompassed by the embodiments of the present disclosure. Moreover, all modes considerable without departing from the essence of disclosure identified by wording described in the claims are encompassed by the embodiments of the present disclosure.

As described above, the present disclosure relates to the breathing function examining instrument for measuring the intraoral pressure of the subject which changes with breathing. The breathing function examining instrument includes the main body portion, the resistance setting unit, and the pressure measurement unit.

The main body portion is shaped in a cylinder and formed with an intake port which is a hole through which an air from an outside flows into the cylinder. One end of the main body portion is provided with the one-way valve for impeding the inflow of air from the outside. In the main body portion, the subject inhales through the other end where one-way valve is not provided.

The resistance setting unit variably sets a magnitude of an inflow resistance to the air flowing into the main body portion. Further, the pressure measurement unit measures a pressure of the air moving in the cylinder of the main body portion as the intraoral pressure. According to the breathing function examining instrument, when the subject implements multiple number of breathing with the prescribed constant ventilation amount with the use of the breathing function examining instrument, if resistors of different magnitudes are set, the multiple number of breathing can be performed with different depths. In other words, according to the breathing function examining instrument, the subject can perform the breathing with different depths in a simpler manner.

As a result, according to the breathing function examining instrument, the intraoral pressure of the subject in breathing having different depths can be easily measured.

Symbols in parenthesis described in the “claims” and “Solution to Issue” represent a correspondence relationship with specific means described in embodiments described later as one aspect, but do not restrict the technical scope of the present disclosure.

Although the present disclosure has been described with reference to the examples, it should be understood that the present disclosure is not limited to the examples or the structures. The present disclosure includes various modification examples and modifications within the equivalent range. In addition, it should be understood that various combinations or aspects, or other combinations or aspects, in which only one element, one or more elements, or one or less elements are added to the various combinations or aspects, also fall within the scope or technical idea of the present disclosure.

What is claimed is:

1. A breathing function examining instrument to measure an intraoral pressure of a subject which changes with breathing, comprising:
a main body portion formed in a cylinder shape, having an intake port as a hole through which air from an outside is to flow into the cylinder, having one end provided with a one-way valve, which is to impede inflow of air from the outside, and having an other end, which is not provided with the one-way valve and through which the subject is to breathe;
a resistance setting unit to variably set a magnitude of an inflow resistance to air, which is to flow into the main body portion; and
a pressure measurement unit[[ (32)]] to measure, as the intraoral pressure, a pressure of air moving in the cylinder of the main body portion,
the resistance setting unit covers at least a part of the intake port to change the magnitude of the inflow resistance,
the resistance setting unit includes an adjustment plate formed in a plate-shape,
the adjustment plate is attached to an outer surface of the main body portion to change an area covering at least a part of the intake port,
the adjustment plate is configured to be rotatable relative to the main body portion,
the adjustment plate is pierced with at least one vent hole, and
the at least one vent hole is smaller in diameter than the intake port and is different in diameter from each other.

2.-5. (canceled)

6. A breathing function examining instrument to measure an intraoral pressure of a subject which changes with breathing, comprising:
a main body portion formed in a cylinder shape, having an intake port as a hole through which air from an outside is to flow into the cylinder, having one end provided with a one-way valve, which is to impede inflow of air from the outside, and having an other end, which is not provided with the one-way valve and through which the subject is to breathe;
a resistance setting unit to variably set a magnitude of an inflow resistance to air, which is to flow into the main body portion; and
a pressure measurement unit to measure, as the intraoral pressure, a pressure of air moving in the cylinder of the main body portion, wherein
the resistance setting unit covers at least a part of the intake port to change the magnitude of the inflow resistance,
the resistance setting unit includes an adjustment plate formed in a plate-shape,
the adjustment plate is attached to an outer surface of the main body portion to change an area covering at least a part of the intake port, and
the adjustment plate has a structure to slide relative to the main body portion to close a part of the intake port.

7. A breathing function examining instrument to measure an intraoral pressure of a subject which changes with breathing, comprising:
a main body portion formed in a cylinder shape, having an intake port as a hole through which air from an outside is to flow into the cylinder, having one end provided with a one-way valve, which is to impede inflow of air from the outside, and having an other end, which is not provided with the one-way valve and through which the subject is to breathe;
a resistance setting unit to variably set a magnitude of an inflow resistance to air, which is to flow into the main body portion; and
a pressure measurement unit to measure, as the intraoral pressure, a pressure of air moving in the cylinder of the main body portion, wherein
the resistance setting unit covers at least a part of the intake port to change the magnitude of the inflow resistance,
the resistance setting unit includes a plurality of fitting portions replaceably fitted to the intake port, and
each of the fitting portions is pierced with a vent hole which is smaller in diameter than the intake port.

8. The breathing function examining instrument according to claim 1, further comprising a flow rate measurement unit to measure an amount of air moving in the cylinder of the main body portion.

9. The breathing function examining instrument according to claim 1, wherein
the main body portion is configured by combining at least two or more parts.

10. The breathing function examining instrument according to claim 1, wherein
an upper limit value of the inflow resistance set with the resistance setting unit is determined as an upper limit value allowing the subject to breathe at resting breathing with a predefined ventilation amount.
